(19) United States
(12) Patent Application Publication
(10) Pub. No.: US 2005/0153938 A1
(43) Pub. Date: Jul. 14, 2005

(54) Polyphosphonate derivatives for toothpaste compositions

(75) Inventors: Benoit Denizot, Angers (FR); Anne Denizot, Angers (FR); Francois Hindre, Rennes (FR); David Portet, Tierce (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Universite D'Angers, ANGERS (FR)

(21) Appl. No.: 10/511,048

(22) PCT Filed: Apr. 14, 2003
(86) PCT No.: PCT/FR03/01178

(30) Foreign Application Priority Data
Apr. 12, 2002 (FR) 02/04596

(51) Int. Cl. A61K 31/663

(52) U.S. Cl. 514/102

(57) ABSTRACT

The invention relates to a medicament containing the polyphosphonate compound of general formula (I) as an active ingredient.

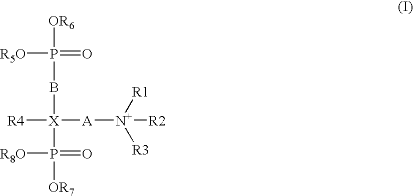

This invention relates to a polyphosphonate compound used as a medicine, particularly for mouth hygiene, a mouth hygiene composition comprising such a biphosphonate compound, use of such a compound for the preparation of a medicine more specifically intended to prevent the appearance and development of dental plaque.

Mouth bacterial flora are composed of many taxons in combination, organised in the form of a multigeneric biofilm commonly called dental plaque. This flora is associated with the development of carious and periodontal pathology. Some species such as Streptococcus mutans, S. sobrinus and Lactobacillus that can be identified in this biofilm produce caries and form primary colonisation species of tooth surfaces by interaction with molecules originating from saliva.

Bacteria species in mouth flora express virulence factors for which some effects can be observed locally and some effects can be observed remotely: colonisation factors (adhesins), persistent factors (aggressins, stress proteins, etc.) and tissular destruction factors (agressins, proteasic activities, etc.). Streptococca in the mouth cavity colonise tooth surfaces by means of adhesins that are proteic constituents of the bacterial wall. S. mutans plays a key role both in triggering and development of the carious process due to its homofermentary type metabolism, and also in particular its capacity to produce glucose homopolymers (soluble dextranes) synthesised by glucosyltransferases (GTFs). GTFs contribute towards the bonding capabilities of carious streptococca by means of glucose polymers.

A caries can affect the enamel, the dentine and the dental pulp of the tooth. Symptoms vary from simple demineralisation to complete destruction of the tooth.

Dental plaque bonds to the very thin layer surrounding each tooth and that includes salivary proteins. A 5-day old dental plaque, if it is not removed by brushing, can form a layer up to about 60 micrometers thick. A caries-producing plaque may often contain 2×10⁸ S. mutans bacteria/mg of dry weight and can quickly release glucose and fructose by fermentation in sufficient quantities to generate an acid pH of the plaque equal to 5.5 or less causing demineralisation of tooth surfaces.

The appearance and development of dental plaque may be inhibited particularly by thorough brushing of the teeth. However, brushing of the teeth is an operation that is tedious if done every day without interruption, requiring strong motivation, a certain amount of skill and good instruction. Moreover, good mouth hygiene by regular brushing is not necessarily sufficient to prevent gingivitis and particularly the formation of dental plaque.

Moreover, many persons are incapable of correctly applying recommended brushing techniques. Moreover, the use of antibiotics is systematically avoided for use in prophylaxis of dental plaque, considering their side effects. Molecules with a bacteriolytic capacity can cause massive release of bacterial constituents or dissemination of antibiotic resistant genes. The role of antiseptics and antibiotics can only be temporary, additional or supplementary.

Some substances have been identified as inhibiting the formation of dental plaque, such as chlorhexidine if used twice a day for mouth rinsing at a concentration of 0.2%. Its prolonged remanence in the mouth cavity makes it more efficient. Chlorhexidine also has an affinity for hydroxyapatite (mineral component of enamel) but due to its side effects (tooth colouring, colouring of the mucous membrane and composites), chlorhexidine is not suitable for a long term treatment.

It has been observed that the use of a varnish containing chlorhexidine associated with thymol (Cervitec©, Vivadent) causes a reduction in caries in sulcuses. This varnish seems particularly suitable for young patients wearing fixed orthodontic appliances.

Chlorhexidine for mouth rinsing is still one of the most frequently recommended antiseptics for the prevention of dental plaque, being indicated when mechanical methods have to be suspended or temporarily reinforced.

Another non-ionic anti-bacterial agent, triclosan, for example mentioned in U.S. Pat. No. 6,136,298, associated with a polyvinyl methyl ether and maleic acid copolymer has the advantage of reducing plaque, gingivitis, tartar and caries, with no side effect and without modifying the ecology of mouth flora. However, a search is continuously being made for new compounds improving the efficiency of treatment against dental plaque.

Another approach is to act on specific interactions that exist between micro-organisms in the mouth and the tooth surfaces. Specific compounds can interfere with these interactions and form a means of prevention or control of the formation of dental plaque. Thus, surfactants or polymers have been used to reduce or prevent salivary proteins from being adsorbed on the enamel surface. Surfactants naturally tend to be adsorbed on all surfaces strongly reducing the interface energy. The affinity of the molecule for an interface is usually affected by the properties of the hydrophobic and hydrophilic parts. Most surfactants are adsorbed on the surface at well-defined orientations, and action on adsorption and desorption of proteins may be intimately associated with the orientation of surfactants on the surface.

There is also a very large number of surfactants that can be used, particularly to improve mouth hygiene, some of which are mentioned in U.S. Pat. No. 6,013,274. For example, a dental composition includes between 0.01 and 20% by weight of a surfactant or an anionic, cationic, non-ionic and zwitterionic mix of surfactants. For example, there are surfactants with an oral tolerance such as monoglycerides, glycerides, sulfonate monoglycerides, other fatty acid ethers, alkyl sulphates, polysorbates, quaternary ammonium alkyl compounds, carboxylates, polyoxyethylenes, phosphonates, sodium alkyl sulphates, sodium sulphate lauryl.

Surfactants are competitively fixed to the tooth surface, thus preventing proteins from bonding to it. However, the difficulty is in identification of a particularly appropriate surfactant with good efficiency for combating dental plaque. Moreover, some surfactants have an excessive foaming effect for the user, as described in U.S. Pat. No. 5,993,784.

The use of molecules that induce precipitation of proteins (precipitants), such as polyionic molecules that displace the tooth protein film and form bridges between and with proteins (for example xanthane gums) are also known.

U.S. Pat. No. 5,270,365 and EP 0 216 681 describe the use of alendronate (4-amino-1-hydroxybutilidene-1,1-biphosphonic acid) either orally or by injection for treatment of parodontitis associated with loss of the dental alveolar bone and against periodontolysis, respectively.

The invention is designed to overcome the disadvantages of prior art by proposing a particularly efficient composition and compound adapted to mouth hygiene, with different possible applications, particularly toothpaste and tooth gels, mouthwashes, chewing gums, non-abrasive gels.

The invention is also intended to provide mouth hygiene compositions using an efficient quantity of active constituent, capable of combating bacterial agents causing alterations to the mouth cavity that can be treated topically.

The invention is designed particularly to obtain a composition including a compound capable of firstly competitively inhibiting attachment of proteins described above, and also efficiently bonding to the tooth surface to be protected, in the long term.

Consequently, the first purpose of the invention is a polyphosphonate compound used as a medicine, the said compound having the general formula I:

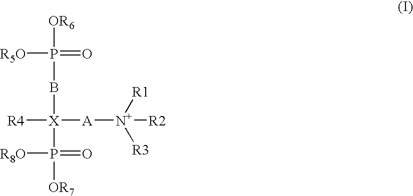

in which:

1) R1, R2, R3, R5, R6, R7, R8 represent an atom of hydrogen or a C1-C6 alkyl or aryl group, independently of each other;

2) X is a carbon C atom or a nitrogen N atom;

3) A, B and C represent a chemical bond, a C1-C6 alkyl or aryl group, a carbonyl group, or a hydrophilic group;

4) R4 represents:

a) either a hydrogen atom, an OH group, a C1-C6 alkyl or aryl group, or a C1-C6 carboxylic acid, a free doublet (if X is a nitrogen N);

b) or a phosphonate with formula: 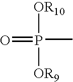
in which R9, R10 represent a hydrogen atom, or a C1-C6 alkyl or aryl group, independently of each other;

c) or a quaternary ammonium group with formula 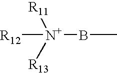
in which R11, R12, R13 represent a hydrogen atom, or a C1-C6 alkyl or aryl group independently of each other, and B represents a chemical bond, a C1-C6 alkyl group, a carbonyl group or a hydrophilic group;

d) or a hydrophilic group;

e) or a polyphosphonate compound with the following general formula II: 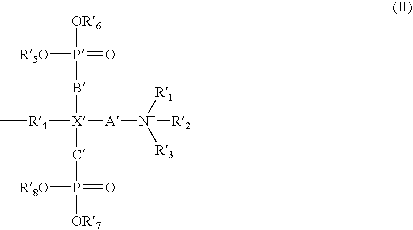
in which:

R′1, R′2, R′3, R′5, R′6, R′7, R′8 represent an atom of hydrogen or a C1-C6 alkyl or aryl group, independently of each other;

X′ is a C atom or an N atom;

A′, B′ and C′ represent a chemical bond, a C1-C6 alkyl or aryl group, a carbonyl group, or a hydrophilic group;

and R′4 represents a C1-C6 alkyl or aryl group, or a C1-C6 carboxylic acid; or a pharmaceutically acceptable salt of these polyphosphonate compounds with formula I or II.

This compound I is intended particularly to inhibit the appearance and development of dental plaque.

Advantageously, this compound I is a biphosphonate compound.

R1, R2, R3 are advantageously identical to each other and represent methyl or ethyl groups.

R5, R6, R7, R8 are advantageously identical to each other and represent hydrogen atoms or methyl groups.

The group 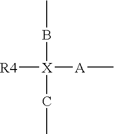
is advantageously a hydrophilic group of 1 to 6 carbon atoms.

The hydrophilic group(s) is (are) typically chosen from among groups with formula —L—Q, in which L is a chemical bond or a C1-C6 alkyl group, linear or ramified and Q is chosen from among:

a) a hydroxyl, amine, carboxyl, sulphate or phosphate group;

b) a linear or ramified C1-C6 alkyl group containing one or several hydroxyl, amine, carboxyl, sulphate, phosphate groups;

c) an M, OM, CONHM, NHCOM group in which M is a hydrophilic group;

d) a hydrophilic group according to points a), b) or c), protected by a group that becomes a hydrophilic group again after a biological hydrolysis.

A preferably represents a C1-C6 alkyl group or a —O—(CH₂)ₙ-group where n=1 to 6, possibly substituted by one or several hydroxyl groups.

According to a second aspect, the purpose of the invention is a mouth composition, advantageously topical and intended for mouth hygiene, comprising a polyphosphonate compound I like that described above, or a mix of such polyphosphonate compounds.

According to one embodiment, the composition comprises between 0.01 and 20%, advantageously between 0.05 and 5%, and even more advantageously between about 0.1 and 2% by weight of compound I.

The composition may also comprise at least one of the elements chosen from among an antibacterial agent, polishing agent, thickening agent, moisturising agent, aroma, sweetening agent, bleaching agent.

The composition is typically in the form of a mouthwash, a spray liquid, a -toothpaste, a tooth gel or similar.

According to another aspect, one purpose of the invention is a polyphosphonate compound I like that described above for making a mouth hygiene composition intended to inhibit the appearance and development of dental plaque.

For the purposes of this invention, the term “alkyl group” means any alkyl group with 1 to 10 carbon atoms, linear or ramified, substituted or not substituted, and particularly the CH₃ group.

For the purposes of this invention, the term “carboxylic acid” means any alkyl group as described above to which a carboxylic group (—COOH) is bonded.

The term “pharmaceutically acceptable” means that salts of the polyphosphonate compound I have the same general pharmacological properties as the free acid form and are acceptable from the toxicity point of view. Possible salts include particularly acetate, benzoate, bicarbonate, bisulphate, bitartrate, borate, bromide, calcium, carbonate, chloride, citrate, lactate, isothionate, malate, methylbromide, methylnitrate, nitrate, ammonium salt, oleate, oxalate, palmitate, phosphate, diphosphate, stearate, sulphate, succinate, tartrate. Furthermore, when the biphosphonate compound includes a part comprising an acid group, pharmaceutically acceptable salts including alkaline metal salts such as sodium or potassium salts, calcium or magnesium salts, or salts formed by appropriate organic binders such as quaternary ammonium salts.

According to one embodiment, this invention relates to a method for treating or preventing the development of dental plaque, comprising administration of such a dentifrice composition, polyphosphonate compounds in a therapeutically efficient quantity, or a pharmaceutically acceptable salt of polyphosphonate. The term “therapeutically efficient quantity” means that the quantity of polyphosphonate administered limits and reduces the appearance and development of dental plaque.

The invention will be particularly well understood after reading the following detailed description.

Bisphosphonate compounds characterised by phosphorus-carbon bonds (P—C—P) are stable compounds resistant to chemical or biological hydrolyses, as mentioned in document WO9836064.

The applicant has successfully demonstrated that the compound I is particularly efficient for preventing bonding of proteins, particularly on tooth surfaces. One particular difficulty lies in identification of compounds with a particularly efficient behaviour for the required application.

The behaviour of polyphosphonate compounds used is complementary to surfactants.

A surfactant is a material that comprises a non-polar hydrophobic part and a polar hydrophilic part, that is capable of forming an interface between two surfaces with different polarities. The surface term is currently used for surfactants and may be a solid surface or a liquid surface or a non-solid surface and a liquid surface. However, the materials that comprise a polar part and a non-polar part are not all efficient surfactants. For example, the non-polar part must be sufficiently large so that it is sufficiently well attached to a non-polar solid surface. The polarity ratio between the hydrophilic part and the hydrophobic part must also be appropriate and optimised for each application.

Non-ionic surfactants are generally defined as being the product of the condensation of alkylene oxide groups (hydrophilic) with a hydrophobic compound that may be aliphatic or aromatic alkyl. For example, we could mention esters of sorbitan polyoxyethylene, fatty alcohol ethoxylates, products derived from condensation of ethylene oxide and the product of the reaction of propylene oxide and diamine ethylene.

Amphoteric surfactants are usually defined as derivatives of secondary and tertiary aliphatic amines in which the aliphatic radical may be linear or ramified, in which one of the aliphatic substitutes has 8 to approximately 18 carbon atoms, and in which one of the aliphatic substitutes comprises an anionic group soluble in water such as a carboxylate, sulfonate, sulphate, phosphate, or phosphonate group. There are also betaines.

Anionic surfactants typically include water soluble salts of alkyl sulphates comprising 8 to 20 carbon atoms in the alkyl radical (for example sodium alkyl sulphate) and water soluble salts of sulfonated monoglycerides of fatty acids with 8 to 20 carbon atoms (for example sodium lauryl sulphate, coconut monoglyceride sulfonates, sarcosinates such as lauroyl sarcosinates, taurates, lauryl sulfoacetates, lauryl isothionates, laureth carboxylates, dodecyl benzenesulfonates.

The applicant has demonstrated that the compound I demonstrated excellent efficiency, this compound comprising at least two phosphonic groups capable of bonding to the surface to be protected, and at least one advantageously trimethyl quaternary ammonium group.

Concerning the number of phosphonic groups, the results are much better with two phosphonic groups than with only one. It would appear that the presence of at least two phosphonic groups can result in at least two anchor points of the compound I on the surface to be protected. Furthermore, the increase in stability will appears to be related to a steric parameter: synergy of two near phosphonate functions provides a better stability for the polyphosphonate compound and particularly the biophosphonate due to the single X atom that separates the two phosphorus atoms.

The applicant has demonstrated that the presence of a quaternary ammonium group very significantly improves bonding results to the surface to be protected. The explanation of this efficiency is not obvious, it would appear that the presence of this quaternary ammonium limits the global content of the compound I that would cause lower repulsion of this compound on the surface, in the event the tooth surface, and therefore to provide protection and give better bonding of the compound on the support.

The compound I may also advantageously comprise:

two phosphonic groups (for good bonding) and two lateral quaternary ammonium chains (for lower repulsion during bonding),

or two phosphonic groups (for good bonding), a quaternary ammonium chain (for lower repulsion during fixation) and a bond with another polyphosphonic compound I, the adjacent compounds I then tending to form a layer distributed on the surface to be protected.

The radical A and/or B of the compound I may an aryl group. The term “aryl group” for the purpose of this invention means one or several aromatic cycles with 5 to 8 carbon atoms, that may be placed adjacent to each other or fused, substituted or not substituted. In particular, the aryl groups may be phenyl groups.

But typically, there is no particular advantage in having more than three carbon atoms or a phenyl radical on substitutes of compound I in order to avoid generating unwanted hydrophobic interactions.

Preferably, A and/or B represent an alkyl group in C1-C3 or an —O—(CH₂)ₙ group where n=1 to 3, possibly substituted by one or several hydroxyl groups.

The carbonaceous chain of compound I may nevertheless typically comprise up to six carbon atoms or hetero-atoms. In order to avoid this type of hydrophobic interaction, the compound I advantageously comprises hydrophilic groups between phosphonic group functions and quaternary ammonium functions of the molecule.

Due to the efficiency of the compound I, a small quantity of this compound in the mouth hygiene composition is enough to be efficient, which results in a saving of the amount of the compound.

Moreover, it is understood that the invention relates to optical isomers of the polyphosphonate compound I.

Tests carried out have demonstrated the high efficiency of polyphosphonate compounds I compared with other compounds, particularly diphosphonic esters, sulfonates, phosphates, carboxylates and diphosphates.

Among biphosphonates, particularly conclusive results have been obtained using 2,2-diphosphono-5-hydroxy-3-oxa-6-hexyltrimethylammonium (TMADP) chloride and 6-trimethylammoniohexyl-1,1-bisphosphonic acid. This TMADP compound has been synthesised in a single step using hydroxyethylidene biphosphonic acid (HEDP) and glycidyltrimethylammonium chloride. The reaction was done in DMSO previously conserved on a molecular sieve. After dissolution of HEDP (2 g; 14.5×10⁻³ mol) at 50° C. in 30 ml of DMSO, the epoxyde (2.2 g; 14.5×10⁻³ mol) is added and the temperature is increased to 110° C. for 24 hours. The DMSO is then evaporated at low pressure and the raw product is dissolved in a minimum amount of distilled water. The TMADP product is then chromatographed on a cation exchanger resin (DOWEX 50WX8-20/50 Mesh, H+form). The column is eluted with distilled water and the pure product is collected at a pH of between 3.0 and 3.5.

The applicant, wishing to make a model explaining bonding of polyphosphonate compounds, has demonstrated in particular that plaque with bonding properties similar to the properties of a tooth surface, such as a stainless steel plate, was efficiently covered and protected against bonding of proteins (contamination by proteins less than 10%) for a concentration of only 2.86×10⁻⁴ mol.I-1 and TMADP after 10 minutes incubation of this plaque with TMAPD (contamination about 15% for a concentration of about 1.4×10⁻⁴ mol.I-1). A sufficient coverage quantity of TMADP molecules on this model is 4×10³ per nm² of plaque surface area.

Compound I is integrated in a composition for mouth hygiene by topical method denoted dentifrice composition typically comprising between 0.01 and 20%, advantageously between 0.05 and 5%, and even better between about 0.1 and 2% by weight of compound I. The term dentifrice means a composition for a topical application on teeth such as a liquid composition (for example a mouthwash and rinsing liquid) or a dentifrice (in the form of a gel, powder or paste).

The composition may be applied to the teeth by various appropriate techniques particularly brushing, staining, spraying, mouthwash. Other possible application means are known to those skilled in the art.

Various other ingredients may be included in the composition, such as prophylactic agents, polishing agents, other surfactants, aromas, appropriate thickening or moisturising agents. It is also important to make sure that these agents do not prevent the required bonding of polyphosphonates on tooth surfaces.

Among the prophylactic agents, it is worth mentioning compounds that limit caries such as sodium fluoride, potassium fluoride, hexylamine hydrofluoride. Typically, these prophylactic agents are present in sufficient quantities to supply a fluoride ion concentration of the order of 0.5 to 2% by weight of the dentifrice composition.

Polishing agents include resins (urea and formaldehyde condensation product), resin particles polymerised by heating (see U.S. Pat. No. 3,070,510), silica xerogels (U.S. Pat. No. 3,538,230), precipitated silica particles, calcium pyrophosphate, insoluble metaphosphate of sodium, hydrated alumina, dicalcium orthophosphate, these agents being sufficiently non-abrasive to not modify the tooth or dentine surface in an unwanted manner. These agents may for example represent 5 to 95% by weight of the dentifrice composition.

Examples of gelation agents or thickening agents include natural gums such as Arabic gum, sodium carboxyl cellulose, hydroxyethyl cellulose, generally representing 0.5 to 10% of the composition by weight of the dentifrice.

When the dentifrice composition is in the form of a mouth liquid, it typically contains an alcohol, a solubilising agent, a non-abrasive cleaning agent, and when it is in the form of a gel it typically includes a thickening agent.

Moisturisers include glycerine, sorbitol, glycol polyethylene and other polyhydric alcohols, these moisturisers representing up to about 35% of the weight of the dentifrice composition. Typically, the dentifrice composition may include a liquid phase representing 10 to 99% by weight and containing water and a moisturising agent in variable proportions.

Aromas may possibly be used in combination with mint oils, menthol, eugenol, orange, lemon, anis, vanillin, thymol, these agents generally representing less than 5% by weight of the dentifrice composition.

The composition may also include sweetening agents (sodium saccharinate), bleaching agents (titanium dioxide or zinc oxide), vitamins, other anti-plaque agents (zinc salts including zinc citrate, copper salts, tin salts, strontium salts, allantoine, chlorhexidine), antibacterial agents (triclosan: 2′, calculus agents (metal pyrophosphates di and/or tetra alkaline), pH adjustment agents, colouring agents, anti-caries agents (casein, urea, glycerophosphates of calcium, sodium fluoride, monosodium fluorophosphate), anti-stain compounds (silicone polymers), anti-inflammatory agents (substituted salicylanilides), desensitising agents (potassium nitrate, potassium citrate). Other agents are mentioned in U.S. Pat. No. 5,258,173.

The pH of the dentifrice composition is typically between 6 and 10.

Example composition for a toothpaste or a tooth gel:

polyphosphonate compound I: 0.2 to 5%

abrasive agent: 10% to 50%

thickening agent: 0.1% to 5%

moisturising agent: 10% to 55%

aroma agent: 0.04% to 2%

sweetening agent: 0.1% to 3%

colouring agent: 0.01% to 0.5%

water: 2% to 45%

Example Composition of Non-Abrasive Gel such as a Subgingival Gel:

polyphosphonate compound I: 0.2% to 5%

thickening agent: 0.1% to 20%

moisturising agent: 10% to 55%

aroma agent: 0.04% to 2%

sweetening agent: 0.1% to 3%

colouring agent: 0.01% to 0.5%

water: 2% to 45%

Example of Mouth Bath Composition:

polyphosphonate compound I: 0.2% to 5%

moisturising agent: 10% to 50%

aroma agent: 0.04% to 2%

sweetening agent: 0.1% to 3%

colouring agent: 0.01% to 0.5%

water: 2% to 45%

ethanol: 0% to 25%.

A dental solution will typically comprise 90 to 99% of water. A chewing gum type composition will typically contain a basic gum (about 50% to 99%), an aroma agent (about 0.4% to 2%), a sweetening agent (about 0.01% to 20%).

Those skilled in the art will easily be able to include different agents as described in U.S. Pat. No. 6,132,702.

For example, the procedure for preparing a dentifrice composition could be as follows: moisturisers such as glycerine, propylene glycol are dispersed with the sweetening agent and water in the mixer, until the mix becomes a homogenous gel. A pigment is then added, together with a pH adjuster if applicable and an anti-cariogenic agent. These ingredients are mixed until a homogenous phase is obtained, to which a polishing agent is then mixed. The mix is then transferred into a high-speed mixer in which a thickening agent, an aroma and compound I are mixed, at a reduced pressure of 20 to 100 mm of Hg. The product obtained is a semi-solid and extrudable paste.

The dentifrice composition is typically applied regularly every day or every two or three days, from one to three times per day, with a pH of about 5 to 9 or 10, and usually between 5.5 and 8.

Synthesis of 6-Trimethylammoniohexyl-1, 1-Bisphosphonic Acid

(CH₃)₃-N+-(CH₂)₅—COOH+H₃PO₃+PCl₃→(CH₃)₃-N+-(CH₂)5-C (PO₃H₂)₂OH.

Procedure

One 6-trimethylammonio caproic acid equivalent and 6 H₃PO₃ equivalents are mixed in a tricol. The mix is heated to 85° C. for 10 minutes. 3 PCl₃ equivalents are added drop by drop and the mix is stirred for two hours. The product obtained is treated with 4 ml of water and is put in the reflux for 12 hours. After passing on activated carbon, the solvent is vacuum evaporated and the reaction product is dissolved in a minimum amount of water. The desired product is obtained after precipitation in excess ethanol.

Efficiency=35%

RMN ¹H, 500 MHz, d(ppm): 3.27 m, CH₂-N⁺(2H); 3.05 s, (CH₃)₃N+(9H); 1.91 m, CH₂-C-P(2H); 1.77 m, CH₂(2H); 1.61 m, CH₂(2H) ; 1.36 m, CH₂(2H)

RMN ³¹H, 500 MHz, d(ppm): 19.44 ppm

The compound obtained may be used in compositions according to this invention.

1. Medicine comprising the polyphosphonate compound with general formula I as active constituent: 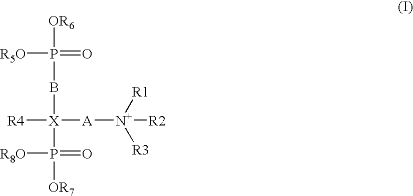
in which:
1) R1, R2, R3, R5, R6, R7, R8 represent an atom of hydrogen or a C1-C6 alkyl or aryl group, independently of each other;
2) X is a carbon C atom or a nitrogen N atom;
3) A represents a C1-C6 alkyl or aryl group, a carbonyl group or a hydrophilic group, B and C represent a chemical bond, a C1-C6 alkyl or aryl group, a carbonyl group, or a hydrophilic group;
4) R4 represents:
a) either a hydrogen atom, an OH group, a C1-C6 alkyl or aryl group, or a C1-C6 carboxylic acid, a free doublet (if X is a nitrogen N);
b) or a phosphonate with formula: 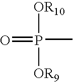
in which R9, R10 represent a hydrogen atom, or a C1-C6 alkyl or aryl group, independently of each other;
c) or a quaternary ammonium group with formula in which:
R′1, R′2, R′3, R′5, R′6, R′7, R′8 represent an atom of hydrogen, or a C1-C6 alkyl or aryl group, independently of each other;
X′ is a C atom or an N atom;
A′, B′ and C′ represent a chemical bond, a C1-C6 alkyl or aryl group, a carbonyl group, or a hydrophilic group;
and R′4 represents a C1-C6 alkyl or aryl group, or a C1-C6 carboxylic acid; or a pharmaceutically acceptable salt of these polyphosphonate compounds with formula I or II,
except for 4-amino-1-hydroxybutilidene-1,1-biphosphonic acid.

2. Medicine according to claim 1, characterised in that R1, R2, R3 are advantageously identical to each other and represent methyl or ethyl groups.

3. Medicine according to either claim 1 or 2, characterised in that R5, R6, R7, R8 are advantageously identical to each other and represent hydrogen atoms or methyl groups.

4. Medicine according to any one of claims 1 to 3, characterised in that the group 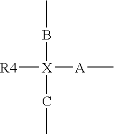
is advantageously a hydrophilic group of 1 to 6 carbon atoms.

5. Medicine according to any one of claims 1 to 4, characterised in that the hydrophilic group(s) is (are) typically chosen from among groups with formula —L—Q, in which L is a chemical bond or a C1-C6 alkyl group, linear or ramified and Q is chosen from among:
a) a hydroxyl, amine, carboxyl, sulphate or phosphate group;
b) a linear or ramified C1-C6 alkyl group containing one or several hydroxyl, amine, carboxyl, sulphate, phosphate groups;
c) an M, OM, CONHM, NHCOM group in which M is a hydrophilic group;
d) a hydrophilic group according to points a), b) or c), protected by a group that becomes a hydrophilic group again after a biological hydrolysis.

6. Medicine according to any one of claims 1 to 6, characterised in that the compound with formula I comprises two phosphonic groups and one quaternary ammonium group.

7. 2,2-diphosphono-5-hydroxy-3-oxa-6-hexyltrimethylammonium chloride for use as a medicine.

8. Composition for mouth hygiene by topical method, characterised in that it comprises a polyphosphonate compound with the following formula I: 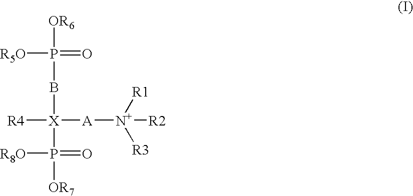
in which:
R1, R2, R3, R4, R5, R6, R7, R8, X, A, B and C are as defined in claim 1,
or one of its pharmaceutically acceptable salts,
or a mix of such polyphosphonate compounds.

9. Composition according to claim 8, characterised in that it comprises between 0.01 and 20%, advantageously between 0.05 and 5%, and even better between about 0.1 and 2% by weight of compound I.

10. Composition according to either claim 8 or 9, characterised in that it also comprises at least one of the elements chosen from among an antibacterial agent, polishing agent, thickening agent, moisturising agent, aroma, sweetening agent, bleaching agent.

11. Composition according to any one of claims 8 to 10, characterised in that it is in the form of a mouthwash, a spray liquid, a toothpaste, a tooth gel.

12. Use of a polyphosphonate compound with formula I: 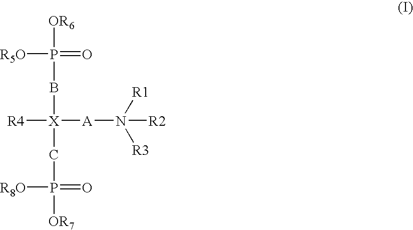
in which R1, R2, R3, R4, R5, R6, R7, R8, X, A, B and C are as defined in claim 1,
or one of its pharmaceutically acceptable salts,
for making a medicine intended to inhibit the appearance and development of dental plaque.

13. Use according to claim 12, characterised in that the compound I is chosen from among:
2,2-diphosphono-5-hydroxy-3-oxa-6-hexyltrimethylammonium chloride and,
6-trimethylammoniohexyl-1,1-bisphosphonic acid.
